(19) United States
(12) Patent Application Publication
(10) Pub. No.: US 2024/0216563 A1
(43) Pub. Date: Jul. 4, 2024

(54) DEVICES AND METHODS FOR MEASURING EFFICACY OF ULTRAVIOLET LIGHT DISINFECTION

(71) Applicant: STRATIX LABS CORPORATION, St. Paul, MN (US)

(72) Inventors: Joshua Erickson, Blaine, MN (US); Mark Mulvahill, St. Louis Park, MN (US)

(73) Assignee: STRATIX LABS CORPORATION, St. Paul, MN (US)

(21) Appl. No.: 18/603,493

(22) Filed: Mar. 13, 2024

Related U.S. Application Data
(63) Continuation of application No. PCT/US2022/043318, filed on Sep. 13, 2022.
(60) Provisional application No. 63/243,853, filed on Sep. 14, 2021.

(51) Int. Cl. A61L 2/28; A61L 2/10; C12M 1/12; C12Q 1/22

(52) U.S. Cl. CPC A61L 2/28; A61L 2/10; C12M 37/06; C12Q 1/22; A61L 2202/11

(57) ABSTRACT

An ultraviolet (UV) light test device is suitable for ascertaining the efficacy of a germicidal UV light source. The UV light test device includes a population of viable microbial material having a residual water content of less than five percent. The population of viable microbial material is encapsulated in a container. At least a portion of the container is formed of a UV light transmissible material such that UV light from the germicidal UV light source is able to contact the population of viable microbial material.

### RELATED APPLICATIONS

This application is a continuation of International Application No. PCT/US2022/043318, filed Sep. 13, 2022, which claims the benefit under 35 USC 119(e) of U.S. Provisional Patent Application Ser. No. 63/243,853 filed Sep. 14, 2021, the disclosures of which are incorporated herein by reference.

### TECHNICAL FIELD

The present disclosure pertains to microbiology. More particularly, the present disclosure pertains to methods and articles for evaluating the antimicrobial efficacy of germicidal ultraviolet light.

### BACKGROUND

Pathogenic microorganisms can have economic, health, and safety impacts.

### SUMMARY

Disclosed herein are test devices for evaluating the antimicrobial efficacy of germicidal ultraviolet light. As an example, an ultraviolet (UV) light test device includes a population of viable microbial material having a residual water content of less than five percent and a container encapsulating the population of viable microbial material, the container formed of a material that is microbe impermeable, at least a portion of the container formed of a UV light transmissible material.

As another example, an antimicrobial light test device may include a population of dried biological material and a container that encapsulates the population of dried biological material. The container may be formed of a material that is microbe impermeable, and at least part of the container may be formed of a material that enables transmission of a germicidal light wavelength. The efficacy of germicidal light may be evaluated or verified against a biological material challenged contained in a germicidal light test device, including any of the test devices described herein.

Alternatively or additionally, the UV light test device may further include an outer package encompassing the container, the outer package formed of an oxygen impermeable material.

Alternatively or additionally, the population of viable microbial material may include between 3 to 9 logs of viable microbial material.

Alternatively or additionally, the population of viable microbial material may have a loss of viability of less than 0.5 logs over at least 30 days when stored at a temperature of 4° C.

Alternatively or additionally, the population of viable microbial material may include bacterial cells or fungal cells.

Alternatively or additionally, the UV light test device may further include a substrate bearing the population of viable microbial material disposed on the substrate.

Alternatively or additionally, the substrate may include a flat surface with a surface area between 10 mm² and 10,000 mm².

Alternatively or additionally, the population of viable microbial material may include a stabilizing material.

Alternatively or additionally, the stabilizing material may include a sugar.

Alternatively or additionally, the stabilizing material may include an antioxidant.

Alternatively or additionally, the stabilizing material may include a growth nutrient.

As another example, an ultraviolet (UV) light test device includes a flat substrate and a population of viable microbial material disposed on the flat substrate, the population of viable microbial material having a residual water content of less than five percent. A container encapsulates the flat substrate bearing the population of viable microbial material and at least a portion of the container formed of a UV light transmissible material.

Alternatively or additionally, the UV light test device may further include an outer package encompassing the container, the outer package formed of an oxygen impermeable material.

Alternatively or additionally, the population of viable microbial material may include between 3 to 9 logs of viable microbial material.

Alternatively or additionally, the population of viable microbial material may have a loss of viability of less than 0.5 logs over at least 30 days when stored at a temperature of 4° C.

Alternatively or additionally, the population of viable microbial material may include bacterial cells or fungal cells.

Alternatively or additionally, the flat substrate may include a flat surface with a surface area between 10 mm² and 10,000 mm².

Alternatively or additionally, the population of viable microbial material may include a stabilizing material.

Alternatively or additionally, the stabilizing material may include a sugar.

Alternatively or additionally, the stabilizing material may include an antioxidant.

Alternatively or additionally, the stabilizing material may include a growth nutrient.

As another example, an ultraviolet (UV) light test device includes a first compartment having a population of viable microbial material disposed on a substrate secured within the first compartment, the population of viable microbial material having a residual water content of less than five percent. A UV light transmissible cover extends over the first compartment such that a UV light from a UV light source is able to contact the population of viable microbial material disposed on the substrate. A second compartment includes a liquid growth media and is sealed from the first compartment. A barrier separates the first compartment and the second compartment and is adapted to be broken after the population of viable microbial material has been exposed to the UV light, the liquid growth media stored within the second compartment able to reach the population of population of viable microbial material once the barrier is broken.

Alternatively or additionally, the barrier may include a frangible member.

Alternatively or additionally, the UV light test device may further include an indicator that provides an indication of whether there is residual viable microbial material after the population of viable microbial material has been exposed to the UV light and contacted with the liquid growth media.

Alternatively or additionally, the population of viable microbial material may include between 3 to 9 logs of viable microbial material.

Alternatively or additionally, the population of viable microbial material may have a loss of viability of less than 0.5 logs over at least 30 days when stored at a temperature of 4° C.

Alternatively or additionally, the population of viable microbial material may include bacterial cells or fungal cells.

Alternatively or additionally, the substrate may include a flat surface with a surface area between 10 mm² and 10,000 mm².

As another example, a method of evaluating antimicrobial efficacy of an ultraviolet (UV) light source includes positioning a UV light test device adjacent to a UV light source, where the UV light test device includes a population of viable microbial material having a residual water content of less than five percent encapsulated within a container formed of a material that is microbe impermeable, at least a portion of the container formed of a UV light transmissible material. Positioning the UV light test device adjacent to the UV light source includes positioning the UV light test device such that UV light from the UV light source is able to pass through the at least a portion of the container formed of the UV light transmissible material. The UV test device is subsequently evaluated to ascertain remaining viability.

Alternatively or additionally, the method may further include removing the UV light test device from an enclosure that is UV light blocking.

Alternatively or additionally, the method may further include contacting the population of viable microbial material with a growth medium subsequent to exposing the population of viable microbial material to the UV light and prior to evaluating the UV test device to ascertain remaining viability.

Alternatively or additionally, evaluating the UV test device to ascertain remaining viability may include a quantitative assessment.

Alternatively or additionally, the quantitative assessment may include determining a loss of viable cells.

Alternatively or additionally, the quantitative assessment may include utilizing a stain or a dye.

Alternatively or additionally, evaluating the UV test device to ascertain remaining viability may include a qualitative assessment.

Alternatively or additionally, the quantitative or qualitative assessment may include evaluating the turbidity of a growth medium.

Alternatively or additionally, the population of viable microbial material may include between 3 to 9 logs of viable microbial material.

Alternatively or additionally, the population of viable microbial material may have a loss of viability of less than 0.5 logs over at least 30 days when stored at a temperature of 4° C.

Alternatively or additionally, the population of viable microbial material may include bacterial cells or fungal cells.

As another example, an ultraviolet (UV) light test device includes an outer container formed of a liquid-impermeable material, with at least a portion of the container including a UV light transmissible barrier. A pre-determined quantity of biological material is coated onto a flat surface of a non-water soluble substrate and the non-water soluble substrate is disposed within the outer container. The biological material on the non-water soluble substrate is orientated to allow UV light from an environment external to the outer container to penetrate directly through the UV light transmissible barrier to the biological material.

Alternatively or additionally, the pre-determined quantity of biological material may include a pre-determined quantity of viable microorganisms.

Alternatively or additionally, the pre-determined quantity of viable microorganisms may include bacterial cells or fungal cells.

Alternatively or additionally, the pre-determined quantity of viable microorganisms may include between 2 to 9 logs colony forming units.

Alternatively or additionally, the pre-determined quantity of viable microorganisms may have a loss of viability of less than 0.5 logs over at least 30 days when stored at a temperature of 4° C.

Alternatively or additionally, the UV transmissible barrier may have a UV transmittance of at least 75%.

Alternatively or additionally, the UV transmissible barrier may have a UV transmittance of at least 90%.

Alternatively or additionally, the UV transmissible barrier may include a fluoropolymer.

Alternatively or additionally, the UV transmissible barrier may include fluoroethylenepropylene.

Alternatively or additionally, the outer container may be impermeable to microorganisms.

Alternatively or additionally, the outer container may be impermeable to gas.

Alternatively or additionally, the outer container may further include an opening that is gas transmissive and microbe impermeable.

Alternatively or additionally, the outer container may include at least two compartments, where the biological material coated onto the flat surface of the non-water soluble substrate is contained in a first compartment with the UV light transmissible barrier extending over the first compartment such that a UV light from a UV light source is able to penetrate to the biological material disposed on the non-water soluble substrate. A second compartment is sealed from the first compartment and includes a liquid growth media. A barrier separates the first compartment and the second compartment and is adapted to be broken after the population of biological material has been exposed to the UV light, the liquid growth media stored within the second compartment able to reach the population of population of viable microbial material once the barrier is broken.

Alternatively or additionally, the barrier may include a frangible member.

Alternatively or additionally, the UV light test device may further include an indicator that provides an indication that viable microorganisms remain in the device following contact with the liquid growth media when the viable microorganisms are exposed to an insufficient exposure of UV light.

Alternatively or additionally, the UV light test device may further include an outer package encompassing the container, the outer package formed of an oxygen impermeable material.

Alternatively or additionally, the biological material may be homogenously coated onto a flat surface of the non-water soluble substrate.

Alternatively or additionally, the biological material may be coated onto a flat surface of the non-water soluble substrate in one or more applications.

Alternatively or additionally, the biological material may be coated onto a flat surface of the non-water soluble substrate as a plurality of spots.

Alternatively or additionally, the non-water soluble substrate may include a flat surface with a surface area between 10 mm² and 10,000 mm².

Alternatively or additionally, the biological material may further include a stabilizing material.

Alternatively or additionally, the stabilizing material may include a sugar.

Alternatively or additionally, the stabilizing material may include an antioxidant.

Alternatively or additionally, the stabilizing material may include a growth nutrient.

Alternatively or additionally, the UV light may pass through a liquid before contacting the biological material.

Alternatively or additionally, the UV light may pass through a liquid before penetrating the UV transmissible barrier.

As another example, a method of evaluating antimicrobial efficacy of an ultraviolet (UV) light source includes positioning a UV light test device adjacent to a UV light source. The UV light test device includes a pre-determined quantity of biological material enclosed within a container formed of a material that is microbe impermeable, at least a portion of the container formed of a UV light transmissible material. Positioning a UV light test device adjacent to a UV light source includes positioning the UV light test device such that UV light from the UV light source is able to pass through the at least a portion of the container formed of the UV light transmissible material. The UV test device is subsequently evaluated to ascertain remaining viability.

Alternatively or additionally, the pre-determined quantity of biological material may include a pre-determined quantity of viable microorganisms.

Alternatively or additionally, the pre-determined quantity of viable microorganisms may include bacterial cells or fungal cells.

Alternatively or additionally, the pre-determined quantity of viable microorganisms may include between 2 to 9 logs colony forming units.

Alternatively or additionally, the pre-determined quantity of viable microorganisms may have a loss of viability of less than 0.5 logs over at least 30 days when stored at a temperature of 4° C.

Alternatively or additionally, the method may further include removing the UV light test device from an enclosure.

Alternatively or additionally, the method may further include contacting the pre-determined quantity of viable microorganisms with a growth medium subsequent to exposing the population of viable microorganisms to the UV light and prior to evaluating the UV test device to ascertain remaining viability.

Alternatively or additionally, evaluating the UV test device to ascertain remaining viability may include a quantitative assessment.

Alternatively or additionally, the quantitative assessment may include determining a loss of viable cells.

Alternatively or additionally, evaluating the UV test device to ascertain remaining viability may include a qualitative assessment.

Alternatively or additionally, the qualitative assessment may include utilizing a stain or a dye.

As another example, a method of evaluating the efficacy of a germicidal light includes removing a germicidal light test device from a sealed container and securing the germicidal light test device in a fixed orientation. The germicidal light test device is exposed to a germicidal light for a duration of time and at a certain distance. The germicidal light test device is removed from the fixed orientation. An analysis is performed to determine the effectiveness of the germicidal light.

Alternatively or additionally, the biological material may be contained within the germicidal light test device and not exposed to the environment.

Alternatively or additionally, performing an analysis may include use of an indicator contained within the germicidal light test device.

Alternatively or additionally, the efficacy of the germicidal light may be evaluated in a non-laboratory setting.

Alternatively or additionally, the efficacy of the germicidal light may be evaluated in a clinical setting.

Alternatively or additionally, the germicidal light test device may be fixed to a surface during exposure to the germicidal light.

Alternatively or additionally, the germicidal light test device may be fixed to an environmental surface during exposure to the germicidal light.

Alternatively or additionally, the method may further include verifying efficacy of a germicidal light disinfection cycle.

Alternatively or additionally, the method may further include verifying an effective distance used in a germicidal light disinfection cycle.

Alternatively or additionally, the method may further include verifying an effective duration of time used in a germicidal light disinfection cycle.

Alternatively or additionally, the method may further include verifying effective placement of a germicidal light used in a germicidal light disinfection cycle.

Alternatively or additionally, the method may further include verifying the effective operation of a germicidal light used in a germicidal light disinfection cycle.

Alternatively or additionally, the method may further include validating the effective operation of a germicidal light used in a germicidal light disinfection cycle.

Alternatively or additionally, the method may further include confirming the effective operation of a germicidal light used in a germicidal light disinfection cycle.

Alternatively or additionally, the method may further include comparing the performance of multiple germicidal lights.

Alternatively or additionally, the method may further include comparing the performance of multiple germicidal light systems.

Alternatively or additionally, the method may further include comparing the performance of multiple germicidal light disinfection cycles.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic block diagram of an illustrative ultraviolet (UV) light test device;

FIG. 2 is a schematic perspective diagram of an illustrative UV light test device;

FIG. 3 is a schematic block diagram of an illustrative UV light test device;

FIG. 4 is a schematic block diagram of an illustrative UV light test device;

FIG. 5 is a schematic block diagram of a test configuration for testing the efficiency of a germicidal UV light source;

FIG. 6 is a schematic diagram of an illustrative UV light test device;

FIG. 7 is a schematic block diagram of an illustrative UV light test device; and

FIG. 8 is a flow diagram showing an illustrative method of evaluating the antimicrobial efficacy of a germicidal UV light source.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure provides methods and articles for evaluating the antimicrobial efficacy of a germicidal UV light source that is to be tested for antimicrobial efficacy against a layer of biological material (e.g., viable microbial material). In some cases, the biological material may be secured relative to a surface such as a flat surface. The articles described herein provide a preserved antimicrobial test article that includes a layer of preserved biological material that is capable of immediate use in efficacy testing of a germicidal UV light source. Ultraviolet (UV) light includes electromagnetic radiation of a variety of wavelengths and includes UV-A, UV-B and UV-C. UV-A has a wavelength that ranges from 315 nanometers (nm) to 400 nm. UV-B has a wavelength that ranges from 280 nm to 315 nm. UV-C has a wavelength that ranges from 100 nm to 280 nm. A number of devices intended for providing germicidal efficacy against a variety of microbes including bacteria and viruses employ lamps that emit UV-C light, among possibly other wavelengths. It has been found that UV-C light is effective against a variety of different microbial species. Other wavelengths outside of the UV-C range have also been found to be effective against a variety of different microbial species.

This disclosure provides inoculated test devices that may be used to test the efficacy of germicidal UV light sources and other germicidal light sources. Currently, preparation of inoculated test articles for disinfectant testing is time-consuming, resource-intensive, and prone to error and variability issues. Furthermore, the current state of the art requires that inoculated test articles be used within a short time frame (e.g., less than two hours) before degradation of microbial viability. These issues contribute to standardization problems and hinder the development of disinfectants.

Provided herein are preserved antimicrobial test articles for evaluating the antimicrobial efficacy of a germicidal UV light source. A preserved antimicrobial test article may include a biological material that is a microorganism, a protein, a nucleic acid (e.g., RNA or DNA) or a molecule that is intended to represent the response of a microorganism to a germicidal UV light source. In some instances, the preserved antimicrobial test article includes a microorganism that is a viable microorganism.

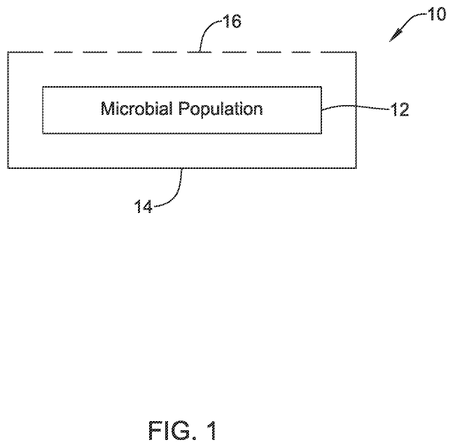

FIG. 1 provides a schematic block diagram of an illustrative UV light test device 10. The illustrative UV light test device 10 includes a population 12 of viable microbial material that has been dehydrated and thus has a residual water content of less than 5 percent. The population 12 of viable microbial material may include at least 0.1 log CFU, at least 0.2 log CFU, at least 0.3 log CFU, at least 0.5 log CFH, at least 1 log CFU, at least 2 log CFU, or at least 3 log CFU. The population 12 of viable microbial material may include 3.0 logs to 9.5 logs (e.g., between 3.0 logs to 9.0 logs, between 3.0 logs to 8.5 logs, between 3.0 logs to 8.0 logs, between 3.0 logs to 7.5 logs, between 3.0 logs to 7.0 logs, between 3.0 logs to 6.5 logs, between 3.0 logs to 6.0 logs, between 3.0 logs to 5.5 logs, between 3.0 logs to 5.0 logs, between 3.0 logs to 4.5 logs, between 3.0 logs to 4.0 logs, between 3.0 logs to 3.5 logs, between 3.5 logs to 9.5 logs between 3.5 logs to 9.0 logs, between 3.5 logs to 8.5 logs, between 3.5 logs to 8.0 logs, between 3.5 logs to 7.5 logs, between 3.5 logs to 7.0 logs, between 3.5 logs to 6.5 logs, between 3.5 logs to 6.0 logs, between 3.5 logs to 5.5 logs, between 3.5 logs to 5.0 logs, between 3.5 logs to 4.5 logs, between 3.5 logs to 4.0 logs, between 4.0 logs to 9.5 logs, between 4.0 logs to 9.0 logs, between 4.0 logs to 8.5 logs, between 4.0 logs to 8.0 logs, between 4.0 logs to 7.5 logs, between 4.0 logs to 7.0 logs, between 4.0 logs to 6.5 logs, between 4.0 logs to 6.0 logs, between 4.0 logs to 5.5 logs, between 4.0 logs to 5.0 logs, between 4.0 logs to 4.5 logs, between 4.5 logs to 9.5 logs, between 4.5 logs to 9.0 logs, between 4.5 logs to 8.5 logs, between 4.5 logs to 8.0 logs, between 4.5 logs to 7.5 logs, between 4.5 logs to 7.0 logs, between 4.5 logs to 6.5 logs, between 4.5 logs to 6.0 logs, between 4.5 logs to 5.5 logs, between 4.5 logs to 5.0 logs, between 5.0 logs to 9.5 logs, between 5.0 logs to 9.0 logs, between 5.0 logs to 8.5 logs, between 5.0 logs to 8.0 logs, between 5.0 logs to 7.5 logs, between 5.0 logs to 7.0 logs, between 5.0 logs to 6.5 logs, between 5.0 logs to 6.0 logs, between 5.0 logs to 5.5 logs, between 5.5 logs to 9.5 logs, between 5.5 logs to 9.0 logs, between 5.5 logs to 8.5 logs, between 5.5 logs to 8.0 logs, between 5.5 logs to 7.5 logs, between 5.5 logs to 7.0 logs, between 5.5 logs to 6.5 logs, between 5.5 logs to 6.0 logs, between 6.0 logs to 9.5 logs, between 6.0 logs to 9.0 logs, between 6.0 logs to 8.5 logs, between 6.0 logs to 8.0 logs, between 6.0 logs to 7.5 logs, between 6.0 logs to 7.0 logs, between 6.0 logs to 6.5 logs, between 6.5 logs to 9.5 logs, between 6.5 logs to 9.0 logs, between 6.5 logs to 8.5 logs, between 6.5 logs to 8.0 logs, between 6.5 logs to 7.5 logs, between 6.5 logs to 7.0 logs, between 7.0 logs to 9.5 logs, between 7.0 logs to 9.0 logs, between 7.0 logs to 8.5 logs, between 7.0 logs to 8.0 logs, between 7.0 logs to 7.5 logs, between 7.5 logs to 9.5 logs, between 7.5 logs to 9.0 logs, between 7.5 logs to 8.5 logs, between 7.5 logs to 8.0 logs, between 8.0 logs to 9.5 logs, between 8.0 logs to 9.0 logs, between 8.0 logs to 8.5 logs, between 8.5 logs to 9.5 logs, or between 8.5 logs to 9.0 logs) of viable microbial material (e.g. colony forming units (CFU)).

Methods for quantifying the amount of viable microbial material are known in the art.

The UV light test device 10 retains a required minimum quantity of biological material for a minimum period of time after the article's date of manufacture. In some examples, the preserved antimicrobial test article retains at least 0.5 log, at least 1 log, at least 1.5 logs, at least 2 logs, at least 2.5 logs, or at least 3 logs (e.g., at least 3.1 logs, at least 3.2 logs, at least 3.3 logs, at least 3.4 logs, at least 3.5 logs, at least 3.6 logs, at least 3.7 logs, at least 3.8 logs, at least 3.9 logs, at least 4.0 logs, at least 4.1 logs, at least 4.2 logs, at least 4.3 logs, at least 4.4 logs, at least 4.5 logs, at least 4.6 logs, at least 4.7 logs, at least 4.8 logs, at least 4.9 logs, at least 5.0 logs, at least 5.1 logs, at least 5.2 logs, at least 5.3 logs, at least 5.4 logs, at least 5.5 logs, at least 5.6 logs, at least 5.7 logs, at least 5.8 logs, at least 5.9 logs, at least 6.0 logs, at least 6.1 logs, at least 6.2 logs, at least 6.3 logs, at least 6.4 logs, at least 6.5 logs, at least 6.6 logs, at least 6.7 logs, at least 6.8 logs, at least 6.9 logs, at least 7.0 logs, at least 7.1 logs, at least 7.2 logs, at least 7.3 logs, at least 7.4 logs, at least 7.5 logs, at least 7.6 logs, at least 7.7 logs, at least 7.8 logs, at least 7.9 logs, at least 8.0 logs, at least 8.1 logs, at least 8.2 logs, at least 8.3 logs, at least 8.4 logs, at least 8.5 logs, at least 8.6 logs, at least 8.7 logs, at least 8.8 logs, at least 8.9 logs, at least 9.0 logs, at least 9.1 logs, at least 9.2 logs, at least 9.3 logs, at least 9.4 logs, or at least 9.5 logs) of viable microbial material (e.g. colony forming units) for a minimum period of time after the article's date of manufacture. In some examples, the minimum period of time after the article's date of manufacture is at least 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, 60 days, 65 days, 70 days, 75 days, 80 days, 85 days, 90 days, 120 days, 150 days, 180 days, 210 days, 240 days, 270 days, 300 days, 330 days, 360 days, or 365 days. In some examples, the minimum period of time after the article's date of manufacture is greater than 365 days.

The microbes used to create the population 12 of viable microbial material can be prokaryotic or eukaryotic organisms. Non-limiting examples of the types of microorganisms that can be included in any of the devices or methods provided herein include: bacteria, fungi, algae, protist, diatom, archaea, and cyanobacteria. In some embodiments, the microorganisms can comprise a single type of microorganism, at least two types of microorganisms, at least three types of microorganisms, at least four types of microorganisms, at least five types of microorganisms, or more than five types of microorganisms.

In some instances, the microorganisms can include a homogenous population of identical cells. In some embodiments, the microorganisms can comprise a heterogeneous population of microbial cells. In some embodiments, the microorganisms can include one organism, at least two different organisms, at least three different organisms, at least four different organisms, at least five different organisms, at least six different organisms, at least seven different organisms, at least eight different organisms, at least nine different organisms, at least ten different organisms, at least twenty different organisms, at least thirty different organisms, or more than thirty different organisms.

In some embodiments, the microorganisms can include one species, at least two species, at least three species, at least four species, at least five species, at least six species, at least seven species, at least eight species, at least nine species, at least ten species, or more than ten species of microorganisms. In some embodiments, the microorganisms can include one strain, at least two strains, at least three strains, at least four strains, at least five strains, at least six strains, at least seven strains, at least eight strains, at least nine strains, at least ten strains, at least fifteen strains, at least twenty strains, at least twenty-five strains, at least thirty strains, at least thirty-five strains, at least forty strains, at least forty-five strains, at least fifty strains, or more than fifty strains of microorganisms. In some examples, the microorganisms include a gram-positive bacteria or a gram-negative bacteria. In some examples, the microorganisms include a fungi (e.g., a yeast). In some examples, the microorganisms can include a spore.

In some embodiments, the microorganism may be selected from the group of: Acetobacter spp., Acetonema spp., Achromobacter spp., Acidithiobacillus spp., Acinetobacter spp., Actinobacillus spp., Actinomyces spp., Aerococcus spp., Aeromonas spp., Aggregatibacter spp., Agrobacterium spp., Alcaligenes spp., Alicyclobacillus spp., Alkalibacillus spp., Alternaria spp., Ammoniphilus spp., Amphibacillus spp. Anaerobacter spp., Anaerospora spp., Anaplasma spp., Aneurinibacillus spp., Anoxybacillus spp., Arcanobacterium spp., Arthrobacter spp., Aspergillus spp., Aureobasidium spp. Azorhizobium spp., Azotobacter spp., Bacillus spp., Bacteroides spp., Bartonella spp. Beggiatoa spp., Bifidobacterium spp., Brevibacterium spp., Brevibacillus spp., Brevundimonas spp., Bordetella spp., Borrelia spp., Brochothrix spp., Brucella spp., Burkholderia spp., Caldanaerobacter spp., Caloramator spp., Calymmatobacterium spp., Caminicella spp., Campylobacter spp., Candida spp., Capnocytophaga spp., Cedecea spp., Cellulosimicrobium spp., Cerasibacillus spp., Chaetomium spp., Chlamydia spp., Chlamydophila spp., Chryseobacterium spp., Citrobacter spp., Cladosporium spp., Clostridioides spp., Clostridium spp., Clostridiisalibacter spp., Cohnella spp., Corynebacterium spp., Coxiella spp., Cronobacter spp., Cryptococcus spp., Curtobacterium spp., Cutibacterium spp., Deinococcus spp., Dendrosporobacter spp., Desulfotomaculum spp., Desulfosporomusa spp., Desulfosporosinus spp., Desulfovibrio spp., Desulfovirgula spp., Desulfunispora spp., Desulfurispora spp., Edwardsiella spp., Eggerthella spp., Ehrlichia spp., Eikenella spp., Elizabethkingia spp., Enterobacter spp., Enterococcus spp., Erysipelothrix spp., Escherichia spp., Eurotium spp., Ferrobacillus spp., Filifactor spp., Filobacillus spp., Finegoldia spp., Fluoribacter spp., Francisella spp., Fusarium spp., Fusobacterium spp., Gallionella spp., Gardnerella spp., Gelria spp., Geobacillus spp., Geosporobacter spp., Geotrichum spp., Gracilibacillus spp., Haemophilus spp., Hafnia spp., Halobacillus spp., Halonatronum spp., Hanseniaspora spp., Helicobacter spp., Heliobacterium spp., Heliophilum spp., Herminiimonas spp., Hormoconis spp., Issatchenkia spp., Klebsiella spp., Kocuria spp., Laceyella spp., Lactobacillus spp., Lactococcus spp., Legionella spp., Lentibacillus spp., Leptothrix spp., Listeria spp., Lysinibacillus spp., Malassezia spp., Mannheimia spp., Mahella spp., Megasphaera spp., Metabacterium spp., Methanobacterium spp., Methylobacterium spp., Meyerozyma spp., Microbacterium spp., Micrococcus spp., Microsporum spp., Moorella spp., Moraxella spp., Morganella spp., Mucor spp., Mycobacterium spp., Mycoplasma spp., Myroides spp., Natroniella spp., Neisseria spp., Nocardia spp., Oceanobacillus spp., Ochrobactrum spp., Oligella spp., Orenia spp., Ornithinibacillus spp., Oxalophagus spp., Oxobacter spp., Paenibacillus spp., Parabacteroides spp., Paraliobacillus spp., Parvimonas spp., Pasteurella spp., Pediococcus spp., Pelospora spp., Pelotomaculum spp., Penicillium spp., Peptostreptococcus spp., Piscibacillus spp., Planifilum spp., Pluralibacter spp., Pneumocystis spp., Pontibacillus spp., Porphyromonas spp., Prevotella spp., Propionibacterium spp., Propionispora spp., Proteus spp., Prototheca spp., Providencia spp., Pseudomonas spp., Ralstonia spp., Raoultella spp., Rhizobium spp., Rhizopus spp., Rhodococcus spp., Rickettsia spp., Rochalimaea spp., Rothia spp., Saccharomyces spp., Salinibacillus spp., Salmonella spp., Salsuginibacillus spp., Seinonella spp., Serratia spp., Shewanella spp., Shigella spp., Shimazuella spp., Sinorhizobium spp., Sphingobacterium spp., Sphingomonas spp., Spirillum spp., Sporacetigenium spp., Sporidiobolus spp., Sporoanaerobacter spp., Sporobacter spp., Sporobacterium spp., Sporohalobacter spp., Sporolactobacillus spp., Sporomusa spp., Sporosarcina spp., Sporotalea spp., Sporotomaculum spp., Staphylococcus spp., Stenotrophomonas spp., Stomatococcus spp., Streptococcus spp., Streptomyces spp., Syntrophomonas spp., Syntrophospora spp., Talaromyces spp., Tenuibacillus spp., Tepidibacter spp., Terribacillus spp., Thalassobacillus spp., Thermoacetogenium spp., Thermoactinomyces spp., Thermoalkalibacillus spp., Thermoanaerobacter spp., Thermoanaerobacterium spp., Thermoanaeromonas spp., Thermobacillus spp., Thermoflavimicrobium spp., Thermovenabulum spp., Thiobacillus spp., Thiothrix spp., Treponema spp., Trichophyton spp., Trichosporon spp., Trueperella spp., Tuberibacillus spp., Ureaplasma spp., Veillonella spp., Vibrio spp., Virgibacillus spp., Viridans spp., Vulcanobacillus spp., Wallemia spp., Wolbachia spp., Yarrowia spp., Yersinia spp., and Zygosaccharomyces spp.

In some embodiments, the microorganism may be selected from the group of: Acetobacter aurantius, Acidithiobacillus thiooxidans, Acinetobacter baumannii (e.g., ATCC® 19606™), Actinomyces israelii, Agrobacterium radiobacter, Agrobacterium tumefaciens, Anaplasma phagocytophilum, Arthrobacter chlorophenolicus, Arthrobacter crystallopoietes, Arthrobacter luteus, Aspergillus brasiliensis (e.g., ATCC® 16404™), Aspergillus flavus (e.g., ATCC® 9643™), Aspergillus fumigatus (e.g., ATCC® 204305™), Aspergillus niger (e.g., ATCC® 6275™), Aspergillus terreus (e.g., ATCC® 1012™), Aureobasidium pullulans, Azorhizobium caulinodans, Azotobacter vinelandii, Bacillus anthracis, Bacillus atrophaeus, Bacillus brevis, Bacillus cereus, Bacillus fusiformis, Bacillus licheniformis, Bacillus megaterium, Bacillus mycoides, Bacillus stearothermophilus, Bacillus subtilis (e.g., ATCC® 6633™), Bacillus thuringiensis, Bacteroides fragilis, Bacteroides gingivalis, Bartonella henselae, Bartonella quintana, Beggiatoa alba, Bordetella bronchiseptica, Bordetella pertussis (e.g., ATCC® 12743™), Borrelia burgdorferi, Brevundimonas diminuta (e.g., ATCC® 19146™), Brucella abortus, Brucella melitensis, Brucella suis, Burkholderia mallei, Burkholderia pseudomallei, Burkholderia cepacia (e.g., ATCC® 25416™), Calymmatobacterium granulomatis, Campylobacter coli, Campylobacter fetus, Campylobacter jejuni (e.g., ATCC® 33291™ and ATCC® 29428™), Campylobacter pylori, Candida albicans (e.g., ATCC® 2091™ and ATCC® 10231™), Candida auris, Candida dubliniensis, Candida krusei, Candida glabrata, Candida lusitaniae, Candida parapsilosis, Candida tropicalis, Chlamydia trachomatis, Chlamydophila pneumoniae, Chlamydophila psittaci, Cladosporium cladosporioides, Cladosporium resinae, Clostridium botulinum, Clostridium difficile (e.g., ATCC® 43598™), Clostridium perfringens, Clostridium sporogenes (e.g., ATCC® 11437™ and ATCC® 19404™), Clostridium tetani, Corynebacterium ammoniagenes, Corynebacterium diphtheriae, Corynebacterium fusiforme, Corynebacterium glutamicum, Corynebacterium stationis, Coxiella burnetii, Cronobacter sakazakii (e.g., ATCC® 12868™), Cryptococcus neoformans, Desulfovibrio africanus, Desulfovibrio desulfuricans, Desulfovibrio salixigens, Desulfovibrio vulgaris, Desulfotomaculum orientis, Desulfotomaculum nigrificans, Ehrlichia chaffeensis, Enterobacter aerogenes (e.g., ATCC® 13048™), Enterobacter cloacae, Enterococcus avium, Enterococcus durans, Enterococcus faecalis (e.g., ATCC® 29212™ and ATCC® 51575™), Enterococcus faecium (e.g., ATCC® 35667™ and ATCC® 51559™), Enterococcus gallinarum, Enterococcus hirae, Enterococcus maloratus, Escherichia coli (e.g., ATCC® 8739™, ATCC® 25922™, ATCC® 10536™, ATCC® 11229™, and ATCC® 35150™), Ferrobacillus ferrooxidans, Fluoribacter bozemanae, Francisella tularensis, Fusobacterium nucleatum, Gallionella ferruginea, Gardnerella vaginalis, Geobacillus stearothermophilus, Haemophilus ducreyi, Haemophilus influenzae (e.g., ATCC® 10211™), Haemophilus parainfluenzae, Haemophilus pertussis, Haemophilus vaginalis, Helicobacter pylori, Hormoconis resinae, Klebsiella oxytoca (e.g., ATCC® 13182™), Klebsiella pneumoniae (e.g., ATCC® 4352™ and ATCC® 51503™), Kocuria rhizophila (e.g., ATCC® 9341™), Lactobacillus acidophilus, Lactobacillus bulgaricus, Lactobacillus casei, Lactobacillus plantarum, Lactococcus lactis, Legionella pneumophila (e.g., ATCC® 33153™), Leptothrix ochracea, Leptothrix discophora, Leptothrix cholodnii, Leptothrix lopholea, Leptothrix mobilis, Listeria monocytogenes (e.g., ATCC® 19117™, ATCC® 19111™, and ATCC® 7644™), Methanobacterium extroquens, Microbacterium multiforme, Micrococcus luteus, Moraxella catarrhalis, Moraxella osloensis, Mycobacterium avium, Mycobacterium bovis (e.g., ATCC® 35743™), Mycobacterium diphtheriae, Mycobacterium intracellulare, Mycobacterium leprae, Mycobacterium lepraemurium, Mycobacterium phlei, Mycobacterium smegmatis, Mycobacterium terrae (e.g., ATCC® 15755™), Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Paenibacillus glucanolyticus, Pasteurella multocida, Pasteurella tularensis, Penicillium chrysogenum, Pluralibacter gergoviae, Pneumocystis carinii, Pneumocystis murina, Porphyromonas gingivalis, Prevotella melaninogenica, Proteus mirabilis (e.g., ATCC® 9240™), Proteus vulgaris, Pseudomonas aeruginosa (e.g., ATCC® 9027™ and ATCC® 15442™), Pseudomonas fluorescens, Pseudomonas putida, Ralstonia pickettii, Rhizobium leguminosarum, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella bongori, Salmonella carrau, Salmonella concord, Salmonella enterica (e.g., ATCC® 10708™, ATCC® 6539™, ATCC® 4931™, ATCC® 13311™, and ATCC® 14028™), Salmonella enteritidis, Salmonella infantis, Salmonella newport, Salmonella schwarzengrund, Salmonella typhi, Salmonella typhimurium, Serratia marcescens (e.g., ATCC® 14756™ and ATCC® 8100™), Shewanella oneidensis, Shewanella putrefaciens, Shigella dysenteriae (e.g., ATCC® 11835™), Shigella flexneri (e.g., ATCC® 29508™), Shigella sonnei (e.g., ATCC® 11060™ and ATCC® 25931™), Sinorhizobium meliloti, Spirillum volutans, Staphylococcus aureus (e.g., ATCC® 6538™, ATCC® 29737™, ATCCR BAA-1683™, and ATCC® 33592™), Staphylococcus epidermidis (e.g., ATCC® 12228™), Staphylococcus hominis, Staphylococcus saprophyticus, Stenotrophomonas maltophilia, Stomatococcus mucilaginous, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus gordonii, Streptococcus lactis, Streptococcus mitior, Streptococcus mitis, Streptococcus mutans, Streptococcus oralis, Streptococcus pneumoniae (e.g., ATCC® 6305™), Streptococcus pyogenes (e.g., ATCC® 12384™ and ATCC® 19615™), Streptococcus rattus, Streptococcus salivarius, Streptococcus sanguis, Streptococcus sobrinus, Thiobacillus concretivorus, Thiobacillus thioparus, Treponema pallidum, Treponema denticola, Trichophyton interdigitale (e.g., ATCC® 9533™), Trichophyton mentagrophytes (e.g., ATCC® 9533™), Trichosporon asahii, Vibrio cholerae (e.g., ATCC® 11623™), Vibrio comma, Vibrio parahaemolyticus, Vibrio vulnificus, Viridans streptococci, Yersinia enterocolitica (e.g., ATCC® 23715™), Yersinia pestis, and Yersinia pseudotuberculosis, including drug-resistant strains thereof. Additional microorganisms that can be included in any of the devices or methods provided herein are contemplated.

The microorganisms may be naturally occurring or genetically modified. In some instances, the microorganism may be genetically modified, e.g., to exhibit fluorescence under certain conditions. The microorganism may be sourced from a laboratory collection or an environmental sample. In some examples, the microorganism is sourced from a preserved collection of reference microorganisms (e.g., ATCC®, NCTC, NCIMB, WDCM, CECT, NCPF, DSM, NBRC, ACM, or IMVS). In other instances, the microorganism is isolated from an environmental sample, e.g., from a human subject or from a surface in a manufacturing facility. Other sources and other methods of collecting and isolating microorganisms are known in the art and may be utilized.

The antimicrobial light test device 10 includes a container 14 that encapsulates the population 12 of viable microbial material. The container 14 may be made of a material that is microbe impermeable in order to prevent escape of any microbial material. The container 14 may be formed of a material that is impermeable to a biological material used in the device in order to prevent escape of any biological material. In some cases, the container 14 may be formed of a material that is impermeable or at least substantially impermeable to liquids such as water, where substantial impermeability may be defined as allowing less than 1 microliter of liquids such as water to pass through over a 24 hour period.

In some cases, the container 14 may be formed of a material that is impermeable or at least substantially impermeable to gases such as oxygen. In some cases, the container 14 may include a vent, port, opening, or other mechanism in order to intentionally enable gas exchange with the environment. A variety of different polymeric materials (e.g., a plastic material) may be used for creating the container 14. In some cases, the container 14 may include materials such as polypropylene, polyethylene, polyethylene terephthalate, glycol-modified polyethylene terephthalate, polystyrene, poly-lactic acid, acrylonitrile butadiene styrene, polyetheretherketone, polyoxymethylene, nylon, polycarbonate, polytetrafluoroethylene, or combinations thereof.

At least a portion of the container 14 may be formed of a material 16 that is enables transmission of a germicidal light. In this, germicidal light transmissibility may be defined as a material that for a given thickness and any other pertinent parameters, allows at least 50 percent of incident light to pass through the material. UV light transmissibility may be defined as a material allowing at least 60 percent of incident light to pass through the material, or at least 70 percent of incident light to pass through the material, or at least 80 percent of incident light to pass through the material, or at least 90 percent of incident light to pass through the material. In some cases, germicidal light transmissibility may vary, depending on the particular wavelength or range of wavelengths of the incident light. Germicidal light transmissibility for a particular material of a particular thickness may be different for varying wavelengths of light.

At least a portion of the container 14 may be formed of a material 16 that is UV light transmissible. In this, UV light transmissibility may be defined as a material that for a given thickness and any other pertinent parameters, allows at least 50 percent of incident UV light to pass through the material. UV light transmissibility may be defined as a material allowing at least 60 percent of incident UV light to pass through the material, or at least 70 percent of incident UV light to pass through the material, or at least 80 percent of incident UV light to pass through the material, or at least 90 percent of incident UV light to pass through the material. In some cases, UV light transmissibility may vary, depending on the particular wavelength or range of wavelengths of the incident UV light. UV light transmissibility for a particular material of a particular thickness may be different for UV-A light than it is for UV-B light or UV-C light, for example.

The material 16 may be any of a variety of different polymeric materials that have a sufficient germicidal light transmissibility (e.g., UV light transmissibility). In some cases, the material 16 may be a fluorinated polymer such as FEP (fluorinated ethylene polymer) or ETFE (ethylene tetrafluoroethylene). In some cases, a fluorinated polymer such as FEP or ETFE having a thickness that ranges from 0.001 inches to 0.1 inches may be used as the material 16. Other suitable materials include quartz. In some cases, at least one side of the container 14 may be formed of the material 16. In some cases, the entire container 14 may be formed of the material 16.

In some examples, the antimicrobial test device may include a mechanism for fixing its orientation during testing of a germicidal light. For example, the antimicrobial test device may include a mechanism for adhering the device to a wall or a surface. The antimicrobial test device may include a mechanism that places the antimicrobial test device in an orientation in which the light transmissive portion of the container is exposed to a germicidal light source.

In some examples after drying the population 12 of viable microbial material can include between about 0% to about 10% (e.g., between 0% to 5%, between 0% to 2.5%, or between 0% to 1%) of residual water content or residual moisture content by weight (e.g., w/w). In some examples, the pre-determined quantity of microorganisms has a viability of 25% or more after 30 days (e.g., 30% of more after 30 days, 40% of more after 30 days, 50% of more after 30 days, 60% of more after 30 days, 70% of more after 30 days, 80% of more after 30 days, 90% of more after 30 days, or 100% of more after 30 days).

In some cases, the coating solution used to deposit the population 12 of viable microbial material onto the substrate 20 may include additional materials such as but not limited to diluents, stabilizing agents, buffer agents, surfactants and the like. Stabilizing agents can be included to improve the stability of the microorganisms through the coating/drying process and over time in storage. In some embodiments, an additive may be included to improve other properties of the device and its use, e.g., homogeneity of the microorganisms coated onto the substrate. Any of the exemplary stabilizing agents provided herein may be included as an additive to modify properties other than stability. Additional additives that can be included to modify properties other than stability are also contemplated.

Non-limiting examples of stabilizing agents include: a sugar (e.g., a monosaccharide, a disaccharide, a reducing sugar, or a non-reducing sugar), a polyol, a polymer (e.g., an oligosaccharide, a polysaccharide, a cellulose-derivative, or a synthetic polymer), an antioxidant, an amino acid, a surfactant, and a buffer.

Non-limiting examples of sugars include: glucose, fructose, xylose, arabinose, sorbose, mannose, rhamnose, galactose, trehalose, maltose, lactose, sucrose, melibiose, maltulose, iso-maltulose, and lactulose.

Non-limiting examples of polymers include: raffinose, stachyose, melezitose, mannotriose, maltodextrin, dextran, starch, inulin, ficoll, alginate, chitosan, methylcellulose, hydroxypropyl cellulose, hydroxyethyl cellulose, hypromellose, xanthan gum, guar gum, pectin, carrageen, galactomannan, gellan gum, cellulose acetate phthalate, carboxy-methyl-cellulose, a salt of alginic acid (e.g., sodium alginate), hydroxyl propyl methyl cellulose, gum acacia, locust bean gum, hydroxyethyl cellulose, hydroxypropyl methylcellulose, polyvinyl alcohol, polyvinyl pyrrolidone, gelatin (e.g., hydrolyzed gelatin and unhydrolyzed gelatin), and polyglycolic acid.

Non-limiting examples of polyols include: sorbitol, arabitol, xylitol, mannitol, erythritol, threitol, and glycerol.

Non-limiting examples of antioxidants include: ascorbic acid, citric acid, acetic acid, a tocopherol, propyl gallate, tertiary butylhydroquinone, butylated hydroxyanisole, and butylated hydroxytoluene.

Non-limiting examples of amino acids include: glycine betaine, sodium glutamate, cysteine, cystine, histidine, and methionine.

Non-limiting examples of buffers include: a potassium phosphate (e.g., monopotassium phosphate), a sodium phosphate (e.g., monosodium phosphate and disodium phosphate), sodium acetate, sodium citrate, sodium succinate, histidine, imidazole, ammonium bicarbonate, a carbonate, [Tris(hydroxymethyl)methylamino]propanesulfonic acid (TAPS), 2-(Bis(2-hydroxyethyl)amino)acetic acid (Bicine), Tris(hydroxymethyl)aminomethane (Tris), 3-[N-Tris(hydroxymethyl)methylamino]-2-hydroxypropanesulfonic acid (Tricine), 3-[N-Tris(hydroxymethyl)methylamino]-2-hydroxypropanesulfonic acid (TAPSO), 4-(2-hydroxyethyl)-1-piperazincethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), Piperazine-N,N′-bis(2-ethanesulfonic acid) (PIPES), Dimethylarsenic acid (Cacodylate), 2-(N-morpholino)ethanesulfonic acid (MES), and N-cyclohexyl-2-aminocthanesulfonic acid (CHES).

Non-limiting examples of surfactants include: a polysorbate (e.g., polysorbate 20, polysorbate 40, polysorbate 60, or polysorbate 80), a poloxamer (e.g., PLURONICS™), Triton X-100, polyethylene glycol, polypropylene glycol, polyethylene glycol/polypropylene glycol block copolymers, polyethylene glycol alkyl ethers, polypropylene glycol alkyl ethers, and polyethylene glycol/polypropylene glycol ether block copolymers.

Non-limiting examples of other stabilizing agents include: milk (e.g., skimmed milk), monosodium glutamate, liquid growth medium, and propylene glycol. Other stabilizing agents are contemplated.

In some instances, the stabilizing mixture may include a sugar. For example, the stabilizing mixture may include a non-reducing sugar such as sucrose. In some instances, the stabilizing mixture may include about 0.1% to about 15% of a stabilizing agent, for example, a sugar or a polyol (e.g., about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%). In some of these and in other instances, the stabilizing mixture may include an antioxidant. In some instances, the stabilizing mixture may include about 0.01% to about 10% of an antioxidant (e.g., about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, or about 0.1% to about 0.5%). In some of these and in other instances, the stabilizing mixture may include ascorbic acid. In some of these and in other instances, the stabilizing mixture may include a surfactant. In some instances, the stabilizing mixture may include about 0.001% to about 2% of a surfactant (e.g., about 0.001% to about 1.9%, about 0.001% to about 1.8%, about 0.001% to about 1.7%, about 0.001% to about 1.6%, about 0.001% to about 1.5%, about 0.001% to about 1.4%, about 0.001% to about 1.3%, about 0.001% to about 1.2%, about 0.001% to about 1.1%, about 0.001% to about 1.0%, about 0.001% to about 0.9%, about 0.001% to about 0.8%, or about 0.001% to about 0.7%, about 0.001% to about 0.6%, about 0.001% to about 0.5%, about 0.001% to about 0.4%, about 0.001% to about 0.3%, about 0.001% to about 0.2%, about 0.001% to about 0.1%, or about 0.001% to about 0.01%). For example, the stabilizing mixture may include a nonionic surfactant such as Triton X-100. In some of these and in other instances, the stabilizing mixture may include an amino acid. In some of these and in other instances, the stabilizing mixture may include a protein. In some of these and in other instances, the stabilizing mixture may include a salt. In some of these and in other instances, the stabilizing mixture may include a polymer. In some of these and in other instances, the stabilizing mixture may include a buffer. For example, the stabilizing mixture may include a phosphate buffer such as phosphate buffered saline. In some of these and in other instances, the stabilizing mixture may include tris(hydroxymethyl)aminomethane. In some of these and in other instances, the stabilizing mixture may include 2-[4-(2,4,4-trimethylpentan-2-yl)phenoxy]ethanol. In some of these and in other instances, the stabilizing mixture may include a growth nutrient. In some of these and in other instances, the stabilizing mixture may include a broth. In one embodiment, the stabilizing mixture includes 8-12% of a sugar (e.g., a non-reducing sugar, e.g., sucrose), 5-7% of an antioxidant (e.g., ascorbic acid), and 0.01-0.1% of a surfactant (e.g., Triton X-100). In an embodiment, the stabilizing mixture includes 10% of a sugar (e.g., a non-reducing sugar, e.g., sucrose), 6% of an antioxidant (e.g., ascorbic acid), and 0.04% of a surfactant (e.g., Triton X-100). In another embodiment, the stabilizing mixture includes between 12-15% of a sugar (e.g., a non-reducing sugar, e.g., sucrose), 5-10% of an antioxidant (e.g., ascorbic acid), and optionally 0.01-0.1% of a surfactant (e.g., Triton X-100).

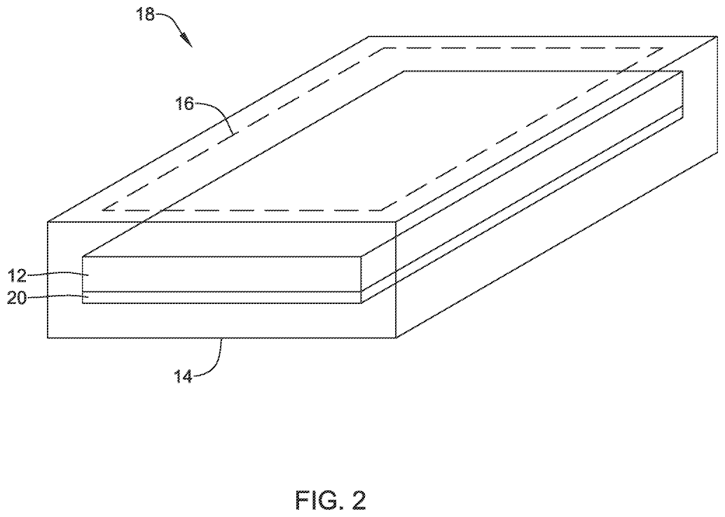

FIG. 2 is a schematic perspective view of a UV light test device 18 that is similar to that shown in FIG. 1, but in FIG. 2 the UV light test device 18 includes a substrate 20 upon which the population 12 of microbial material has been deposited. In some cases, the population 12 of microbial material may be deposited on the substrate 20 as a monolayer or substantially a monolayer in order to permit UV light passing through the material 16 to reach as many individual microbes as possible. While the UV light test device 18 is illustrated as being rectilinear, it will be appreciated that this is merely illustrative, as the UV light test device 18 may take any of a variety of shapes.

In some cases, the substrate 20 is flat in order to maximize the population 12 that is exposed to UV light when the UV light test device 18 is used. The substrate 20 may be formed of any suitable material. In some cases, the substrate 20 may be formed of a polymeric material such as polypropylene, for example. The substrate 20 may be square, rectilinear, round or ovoid, for example. The substrate 20 may have a surface area between 10 mm² to 10,000 mm² (e.g., between 10 mm² to 9,000 mm², between 10 mm² to 8,000 mm², between 10 mm² to 7,000 mm², between 10 mm² to 6,000 mm², between 10 mm² to 5,000 mm², between 10 mm² to 4,000 mm², between 10 mm² to 3,000 mm², between 10 mm² to 2,000 mm², between 10 mm² to 1,000 mm², between 10 mm² to 500 mm², between 10 mm² to 100 mm², between 10 mm² to 50 mm², between 1,000 mm² to 10,000 mm², between 1,000 mm² to 9,000 mm², between 1,000 mm² to 8,000 mm², between 1,000 mm² to 7,000 mm², between 1,000 mm² to 6,000 mm², between 1,000 mm² to 5,000 mm², between 1,000 mm² to 4,000 mm², between 1,000 mm² to 3,000 mm², between 1,000 mm² to 2,000 mm², 1,000 mm² to 1,500 mm², between 1,500 mm² to 3,000 mm², between 1,500 mm² to 2,500 mm², between 1,500 mm² to 2,000 mm², between 2,000 mm² to 10,000 mm², between 2,000 mm² to 9,000 mm², between 2,000 mm² to 8,000 mm², between 2,000 mm² to 7,000 mm², between 2,000 mm² to 6,000 mm², between 2,000 mm² to 5,000 mm², between 2,000 mm² to 4,000 mm², between 2,000 mm² to 3,000 mm², between 2,000 mm² to 2,500 mm², between 2,500 mm² to 4,000 mm², between 2,500 mm² to 3,500 mm², between 2,500 mm² to 3,000 mm², between 3,000 mm² to 10,000 mm², between 3,000 mm² to 9,000 mm², between 3,000 mm² to 8,000 mm², between 3,000 mm² to 7,000 mm², between 3,000 mm² to 6,000 mm², between 3,000 mm² to 5,000 mm², between 3,000 mm² to 4,000 mm², between 3,000 mm² to 3,500 mm², between 3,500 mm² to 5,000 mm², between 3,500 mm² to 4,500 mm², between 3,500 mm² to 4,000 mm², between 2,500 mm² to 3,000 mm², between 4,000 mm² to 10,000 mm², between 4,000 mm² to 9,000 mm², between 4,000 mm² to 8,000 mm², between 4,000 mm² to 7,000 mm², between 4,000 mm² to 6,000 mm², between 4,000 mm² to 5,000 mm², between 4,000 mm² to 4,500 mm², between 4,500 mm² to 6,000 mm², between 4,500 mm² to 5,500 mm², between 4,500 mm² to 5,000 mm², between 5,000 mm² to 6,000 mm², between 6,000 mm² to 7,000 mm², between 7,000 mm² to 8,000 mm², between 8,000 mm² to 9,000 mm², between 9,000 mm² to 10,000 mm², or between 9,500 mm² to 10,000 mm²).

The substrate 20 may have a flat side with a surface area between 10 mm² to 10,000 mm² (e.g., between 10 mm² to 9,000 mm², between 10 mm² to 8,000 mm², between 10 mm² to 7,000 mm², between 10 mm² to 6,000 mm², between 10 mm² to 5,000 mm², between 10 mm² to 4,000 mm², between 10 mm² to 3,000 mm², between 10 mm² to 2,000 mm², between 10 mm² to 1,000 mm², between 10 mm² to 500 mm², between 10 mm² to 100 mm², between 10 mm² to 50 mm², between 1,000 mm² to 10,000 mm², between 1,000 mm² to 9,000 mm², between 1,000 mm² to 8,000 mm², between 1,000 mm² to 7,000 mm², between 1,000 mm² to 6,000 mm², between 1,000 mm² to 5,000 mm², between 1,000 mm² to 4,000 mm², between 1,000 mm² to 3,000 mm², between 1,000 mm² to 2,000 mm², 1,000 mm² to 1,500 mm², between 1,500 mm² to 3,000 mm², between 1,500 mm² to 2,500 mm², between 1,500 mm² to 2,000 mm², between 2,000 mm² to 10,000 mm², between 2,000 mm² to 9,000 mm², between 2,000 mm² to 8,000 mm², between 2,000 mm² to 7,000 mm², between 2,000 mm² to 6,000 mm², between 2,000 mm² to 5,000 mm², between 2,000 mm² to 4,000 mm², between 2,000 mm² to 3,000 mm², between 2,000 mm² to 2,500 mm², between 2,500 mm² to 4,000 mm², between 2,500 mm² to 3,500 mm², between 2,500 mm² to 3,000 mm², between 3,000 mm² to 10,000 mm², between 3,000 mm² to 9,000 mm², between 3,000 mm² to 8,000 mm², between 3,000 mm² to 7,000 mm², between 3,000 mm² to 6,000 mm², between 3,000 mm² to 5,000 mm², between 3,000 mm² to 4,000 mm², between 3,000 mm² to 3,500 mm², between 3,500 mm² to 5,000 mm², between 3,500 mm² to 4,500 mm², between 3,500 mm² to 4,000 mm², between 2,500 mm² to 3,000 mm², between 4,000 mm² to 10,000 mm², between 4,000 mm² to 9,000 mm², between 4,000 mm² to 8,000 mm², between 4,000 mm² to 7,000 mm², between 4,000 mm² to 6,000 mm², between 4,000 mm² to 5,000 mm², between 4,000 mm² to 4,500 mm², between 4,500 mm² to 6,000 mm², between 4,500 mm² to 5,500 mm², between 4,500 mm² to 5,000 mm², between 5,000 mm² to 6,000 mm², between 6,000 mm² to 7,000 mm², between 7,000 mm² to 8,000 mm², between 8,000 mm² to 9,000 mm², between 9,000 mm² to 10,000 mm², or between 9,500 mm² to 10,000 mm²).

Other examples of substrate material, shape, dimensions, and other features are contemplated. As can be appreciated by one skilled in the art, a certain substrate can be selected depending on the specific embodiment and application.

In some embodiments, the population 12 of viable microbial material may be deposited onto the substrate 20 by applying a volume of between 1 to 200 microliters of a coating solution may be applied onto the substrate 20. In some embodiments, 5 microliters or less (e.g., 4 microliters or less, 3 microliters or less, 2 microliters or less, 1 microliters or less, 900 nanoliters or less, 800 nanoliters or less, 700 nanoliters or less, 600 nanoliters or less, 500 nanoliters or less, 400 nanoliters or less, 300 nanoliters or less, 200 nanoliters or less, 100 nanoliters or less, 50 nanoliters or less, 25 nanoliters or less, 10 nanoliters or less, 1 nanoliter or less, 900 picoliters or less, 800 picoliters or less, 700 picoliters or less, 600 picoliters or less, 500 picoliters or less, 400 picoliters or less, 300 picoliters or less, 200 picoliters or less, 100 picoliters or less, 50 picoliters or less, 25 picoliters or less, or 10 picoliters or less) of the coating solution may be applied onto the substrate 20. In some instances, the volume of coating solution may be applied to the substrate 20 in a single application or by multiple applications. In some instances, each application of the coating solution to the substrate 20 may include any of the exemplary volumes provided herein. For example, the coating solution may be applied to the substrate 20 in multiple applications (e.g., between 2 applications to 1000 applications) each including 5 microliters or less of coating solution.

In some instances, a spreading device may be used to spread the coating solution along the substrate 20. However, spreading is not required. In some instances, the coating solution may be applied to the substrate 20 as a plurality of droplets, spots, or microbial clusters. In some instances, the coating solution may be applied as a series or array of droplets, spots, or microbial clusters. The coating solution may be allowed to air dry (e.g., dry at room temperature). In some instances, the coating solution may be dried under vacuum and/or a controlled pressure. In some instances, the coating solution may be dried under a modified and/or controlled temperature.

The coating solution may be allowed to dry for a suitable time period such as 1-60 minutes. In some embodiments of any of the devices, apparatuses, and inoculating systems provided herein, the coating solution may be allowed to dry for 1-50 minutes, 1-45 minutes, 1-40 minutes, 1-35 minutes, 1-30 minutes, 1-25 minutes, 1-20 minutes, 1-15 minutes, 1-10 minutes, 1-5 minutes, or 1 minute or less. In some embodiments, the coating solution may be allowed to dry at room temperature. In other embodiments, the coating solution may be allowed to dry at an elevated temperature (e.g., between 25° C. to 40° C.). In some instances, the coating solution may be subject to vacuum drying. In some instances, the coating solution is dried in a process that does not include freezing the coating solution. For example, the coating solution may be dried in a process that does not include freeze drying or lyophilization. In other instances, the coating solution may be dried using lyophilization.

In some cases, a suitable volume of the stabilizing mixture mixed with the known quantity/concentration of microorganisms (e.g., which may be referred to as the coating solution or mixture) may be disposed on the substrate 20. For example, 1-10 microliters of the coating solution may be disposed onto the substrate 20. This may include pipetting the coating solution onto the substrate 20.

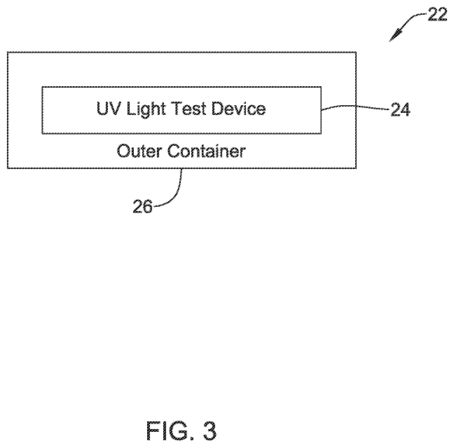

FIG. 3 is a schematic block diagram of an illustrative assembly 22 that includes a UV light test device 24 disposed within an outer container 26. The UV light test device 24 may be considered as being an example of the UV light test device 10 (FIG. 1) or the UV light test device 18 (FIG. 2). The outer container 26 may be configured to protect the UV light test device 24 from exposure to the environment. In particular, the outer container 26 may be formed of a material such as a polymeric material that effectively blocks UV light. As such, the outer container 26 prevents UV light from reaching the microbial population held within the UV light test device 24 until such time as the UV light test device 24 is removed from the outer container 26 in order to use the UV light test device 24 to test the efficacy of a germicidal UV light source.

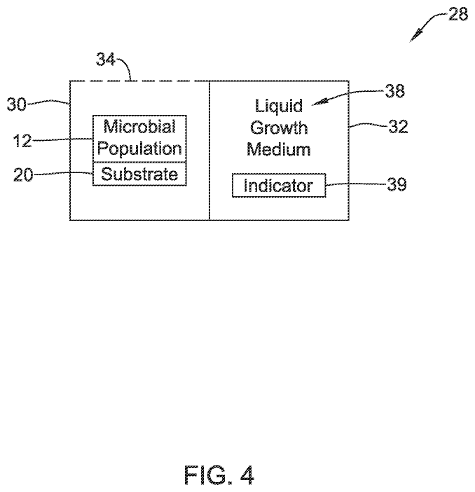

FIG. 4 is a schematic block diagram showing an illustrative UV test device 28 that includes a first container 30 and a second container 32. The first container 30 includes the population 12 of viable microbial material. In some cases, as shown, the population 12 of viable microbial material is disposed on the substrate 20. The second container includes a liquid growth medium 38 that may be brought into contact with the population 12 of viable microbial material after the population 12 of viable microbial material has been exposed to UV light. The liquid growth medium 38, once it contacts the population 12 of viable microbial material, may serve to facilitate growth and replication of any microbes within the population 12 of viable microbial material that remains viable after the UV light exposure. Various methods may be utilized to ascertain whether there is any growth or replication of any microbes, and thus ascertain the efficacy of the UV light to which the population 12 of viable microbial material was exposed to.

In some cases, the UV test device 28 may include an indicator 39 that may be used to ascertain the existence of viable microbial material after exposure to the UV light. The indicator 39 may take a variety of forms, and may or may not be physically disposed within the second container 32. As an example, the indicator 39 may include a dye or other substance that is able to bind with viable microbial material in order to illustrate the presence of viable microbial material. In some cases, the indicator may produce a result within 24 hours, 18 hours, 12 hours, 6 hours, 2 hours, 1 hour, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, 1 minute or less than 1 minute after exposure to the germicidal light. In some cases, the indicator is a rapid readout. In some cases, the indicator is an enzymatic readout.

The first container 30 includes a cover 34 that is formed of a material that is UV light transmissible. UV light transmissibility may be defined as a material that for a given thickness and any other pertinent parameters, allows at least 50 percent of incident UV light to pass through the material. UV light transmissibility may be defined as a material allowing at least 60 percent of incident UV light to pass through the material, or at least 70 percent of incident UV light to pass through the material, or at least 80 percent of incident UV light to pass through the material, or at least 90 percent of incident UV light to pass through the material. In some cases, UV light transmissibility may vary, depending on the particular wavelength or range of wavelengths of the incident UV light. UV light transmissibility for a particular material of a particular thickness may be different for UV-A light than it is for UV-B light or UV-C light, for example. The material 16 may be any of a variety of different polymeric materials that have a sufficient UV light transmissibility. In some cases, the material 16 may be a material that has sufficient transmissibility of the germicidal light wavelength (e.g., a wavelength outside the UV spectrum). In some cases, the material 16 may be a fluorinated polymer such as FEP (fluorinated ethylene polymer) or ETFE (ethylene tetrafluoroethylene). In some cases, a fluorinated polymer such as FEP or ETFE having a thickness that ranges from 0.001 inches to 0.1 inches may be used as the material 16. Other suitable materials include quartz.

The UV test device 28 includes a barrier 36 that seals the first container 30 from the second container 32 and thus prevents the liquid growth medium 38 from prematurely contacting the population 12 of viable microbial material. In some cases, the barrier 36 may be formed of a material that may be ruptured, penetrated or otherwise broken in order to allow the liquid growth medium 38 to reach and contact any viable microbes remaining of the population 12 of viable microbial material.

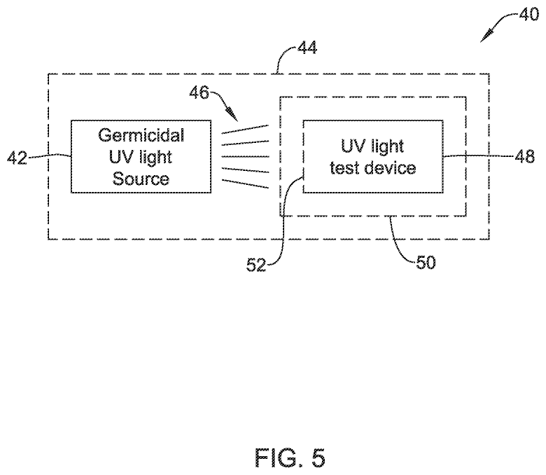

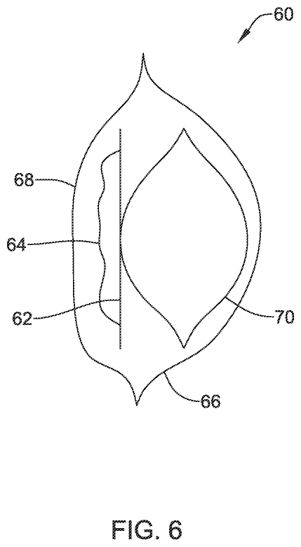

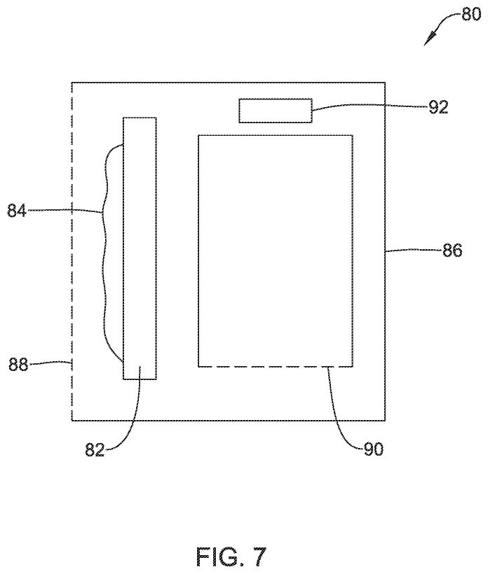

FIG. 5 is a schematic block diagram of an illustrative test configuration 40. The test configuration 40 includes a germicidal UV light source 42. The germicidal UV light source 42 may be configured to reduce or eliminate microbial activity within a space 44. The space 44 may generally represent a room, for example, or perhaps a portion of a larger space. The germicidal UV light source 42 may include one or more UV lamps (not shown) that are configured to output UV light 46. A UV light test device 48, which may be considered as representative of the UV light test device 10, the UV light test device 18, the UV light test device 28, the UV light test device 60 (to be discussed with respect to FIG. 6) or the UV light test device 80 (to be discussed with respect to FIG. 7) may be removed from a protective container 50 and placed such that the UV light 46 passes through a UV light transmissible portion 52 of the UV light test device 48 and can thus impact the microbial material encapsulated within the UV light test device 48.

In some cases, the UV light from the germicidal UV light source 42 passes through a liquid before contacting the biological material within the UV light test device 48. In some instances, the UV light from the germicidal UV light source 42 passes through a liquid before penetrating the UV transmissible barrier of the UF light test device 48.

FIG. 6 is a schematic diagram of an illustrative UV light test device 60. The UV light test device 60 includes a substrate 62. The substrate 62 may, for example, be a non-water soluble substrate. A microbial challenge 64 may be disposed on the substrate 62. In some cases, the microbial challenge 64 may be disposed on a single side of the substrate 62 and may be at least substantially a single layer in order to maximize UV exposure to the microbial challenge 64. The substrate 62 and the microbial challenge 64 are disposed within an outer container 66. At least a portion 68 of the outer container 66 may be formed of a UV transmissible material such that UV light that is incident upon the outer container 66 is able to pass through the portion 68 and impinge on the microbial challenge 64. In some cases, an entirety of the outer container 66 may be formed of a UV transmissible material. Examples of suitable UV transmissible materials include a fluorinated polymer such as FEP (fluorinated ethylene polymer) or ETFE (ethylene tetrafluoroethylene). In some cases, a fluorinated polymer such as FEP or ETFE having a thickness that ranges from 0.001 inches to 0.1 inches may be used. Other suitable materials include quartz.

The UV light test device 60 includes a frangible pouch 70 that includes a growth medium in it. After the microbial challenge 64 has been exposed to a suitable source of UV light for a desired length of time, a user can squeeze the outer container 66 and thereby provide a force to the frangible pouch 70, causing the frangible pouch 70 to burst. As a result, the growth medium, which may be a liquid, is able to contact the microbial challenge 64. In some cases, the growth medium will permit any remaining viable microbial material to grow and thus be more easily detected. While not shown, in some cases the UV light test device 60 may be packaged within an outer packaging that protects the UV light test device 60 from environmental intrusions, such as but not limited to oxygen and UV light present in sunlight, for example.

FIG. 7 is a schematic diagram of an illustrative UV light test device 80. The UV light test device 80 includes a substrate 82. The substrate 82 may, for example, be a non-water soluble substrate. A microbial challenge 84 may be disposed on the substrate 82. In some cases, the microbial challenge 84 may be disposed on a single side of the substrate 82 and may be at least substantially a single layer in order to maximize UV exposure to the microbial challenge 84. The substrate 82 and the microbial challenge 84 are disposed within an outer container 86. At least a portion 88 of the outer container 86 may be formed of a UV transmissible material such that UV light that is incident upon the outer container 86 is able to pass through the portion 88 and impinge on the microbial challenge 84. In some cases, an entirety of the outer container 86 may be formed of a UV transmissible material. Examples of suitable UV transmissible materials include a fluorinated polymer such as FEP (fluorinated ethylene polymer) or ETFE (ethylene tetrafluoroethylene). In some cases, a fluorinated polymer such as FEP or ETFE having a thickness that ranges from 0.001 inches to 0.1 inches may be used. Other suitable materials include quartz.

The UV light test device 80 includes a frangible sealed pouch 90 that includes a growth medium in it. After the microbial challenge 84 has been exposed to a suitable source of UV light for a desired length of time, a user can squeeze the outer container 86 and thereby provide a force to the frangible sealed pouch 90, causing the frangible sealed pouch 90 to burst. As a result, the growth medium, which may be a liquid, is able to contact the microbial challenge 84. In some cases, the growth medium will permit any remaining viable microbial material to grow and thus be more easily detected. The UV light test device 80 includes an opening 92 that may be used, for example, in manufacturing the UV light test device 80, such as adding the microbial challenge 84 to the UV light test device 80. In some cases, the opening 92, which may also be considered to be a port, may be gas transmissive yet microbe impermeable. While not shown, in some cases the UV light test device 80 may be packaged within an outer packaging that protects the UV light test device 80 from environmental intrusions, such as but not limited to oxygen and UV light present in sunlight, for example.

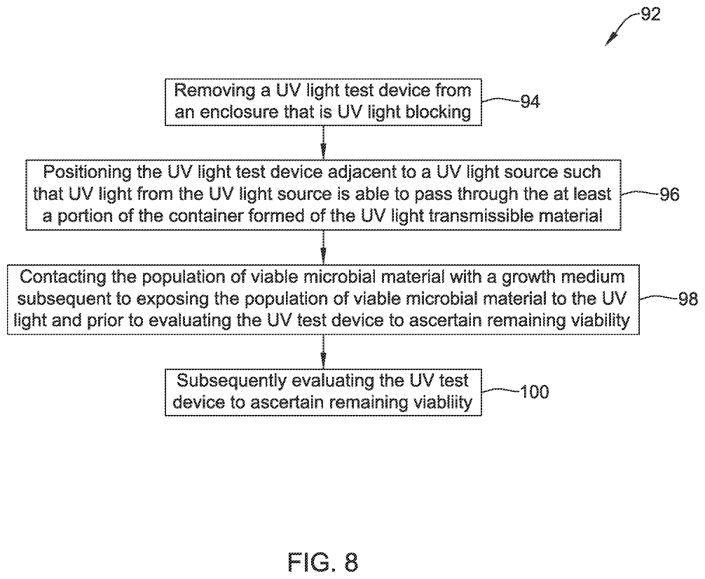

FIG. 8 is a flow diagram showing an illustrative method 92 of evaluating the antimicrobial efficacy of an ultraviolet (UV) light source. The method 92 optionally includes removing a UV light test device from an enclosure that is UV light blocking, as indicated at block 94. In some cases, the UV light test device includes a population of viable biological material such as but not limited to microbial material having a residual water content of less than five percent encapsulated within a container formed of a material that is microbe impermeable, at least a portion of the container formed of a UV light transmissible material. In some cases, the UV light test device includes a predetermined quantity of biological material. The UV light test device may include a predetermined quantity of microbial material, for example. As an example, the population of viable microbial material may include between 2 or 3 to 9 logs of viable microbial material. The population of viable microbial material may be sufficiently stable such that the population of viable microbial material has a loss of viability of less than 0.5 logs over at least 30 days when stored at a temperature of 4° C. The population of viable microbial material may include bacterial cells or fungal cells.

The UV light test device is positioned adjacent to a UV light source includes positioning the UV light test device such that UV light from the UV light source is able to pass through the at least a portion of the container formed of the UV light transmissible material, as indicated at block 96. In some cases, the population of viable microbial material is contacted with a growth medium subsequent to exposing the population of viable microbial material to the UV light and prior to evaluating the UV test device to ascertain remaining viability, as indicated at block 98. The UV test device is subsequently evaluated to ascertain remaining viability, as indicated at block 100. In some cases, evaluating the UV test device to ascertain remaining viability includes a quantitative assessment such as but not limited to determining a loss of viable cells. In some cases, evaluating the UV test device to ascertain remaining viability includes a qualitative assessment. As an example, a qualitative assessment may include utilizing a stain or a dye.

In some instances, a method of evaluating the efficacy of a germicidal light includes removing a germicidal light test device from a sealed container and securing the germicidal light test device in a fixed orientation. The germicidal light test device is exposed to a germicidal light for a duration of time and at a certain distance. The germicidal light test device is removed from the fixed orientation. An analysis is performed to determine the effectiveness of the germicidal light. The biological material may be contained within the germicidal light test device and not exposed to the environment. Performing an analysis may include use of an indicator contained within the germicidal light test device.

In some cases, the efficacy of the germicidal light may be evaluated in a non-laboratory setting. In some cases, the efficacy of the germicidal light may be evaluated in a clinical setting. It will be appreciated that evaluating the efficacy of the germicidal light may include ascertaining whether the germicidal light was sufficient to kill at least a substantial fraction of the biological material being used to test the efficacy of the germicidal light, including killing at least 10 percent, at least 20 percent, at least 30 percent, at least 40 percent, at least 50 percent, at least 60 percent, at least 70 percent, at least 80 percent, at least 90 percent, or more, of the biological material present in the test device. The germicidal light test device may be fixed to a surface during exposure to the germicidal light, for example. The germicidal light test device may be fixed to an environmental surface during exposure to the germicidal light.

In some cases, the method may further include verifying efficacy of a germicidal light disinfection cycle, meaning that the germicidal light disinfection cycle killed a substantial fraction of the biological material present. In some cases, the method may further include verifying an effective distance used in a germicidal light disinfection cycle. In some cases, the method may further include verifying an effective duration of time used in a germicidal light disinfection cycle. This means that the germicidal light was at an effective distance, and/or was operated for a sufficient period of time, in order to kill a substantial fraction of the biological material present.

The method may further include verifying effective placement of a germicidal light used in a germicidal light disinfection cycle, for example. In some cases, the method may further include verifying the effective operation of a germicidal light used in a germicidal light disinfection cycle. In some cases, the method may further include validating the effective operation of a germicidal light used in a germicidal light disinfection cycle. In some cases, the method may further include confirming the effective operation of a germicidal light used in a germicidal light disinfection cycle. Efficacy may be determined by ascertaining whether a substantial fraction of the biological material that was present is killed. In some cases, the method may further include comparing the performance of multiple germicidal lights. The method may further include comparing the performance of multiple germicidal light systems, and/or comparing the performance of multiple germicidal light disinfection cycles.

### EXAMPLES

### Prophetic Example 1—FEP Bag Device

A test device for monitoring germicidal ultraviolet light efficacy against microorganisms can be prepared in the following way: Staphylococcus aureus (ATCC 6538) can be grown in synthetic broth for approximately 24 hours at 35° C. S. aureus cells can be diluted into a sterile coating solution containing sucrose (0.4% w/v), ascorbic acid (0.5%), casamino acids (0.6%), and triton X100 (0.04%) to yield approximately 3.0×10\hat{ }6 CFU/carrier. The coating solution can then be spotted onto a sterile polypropylene carrier (1″×1″) by spotting 10, 1 μL droplets in an array pattern over the surface of the carrier. The carrier can then be air dried for 20 minutes at atmospheric pressure and approximately 30% relative humidity.

The coated carrier can be placed in a sterile polypropylene cartridge (1″×3″) that holds the carrier. The cartridge containing the carrier can then be placed into a sterile fluorinated ethylene propylene (FEP) bag (approximately 3.5″×2″) that has a small vent (5 mm diameter hole) on the side of the bag opposite to the coated side of the carrier to allow gas exchange with the package environment. The bag can then placed in a mylar foil pouch with a desiccant and oxygen scavenger and stored at 2-8 C.

At the point of UV testing, the mylar pouch may be removed from 2-8 C storage. The mylar foil pouch can be opened and the FEP bag can be removed from the mylar foil pouch. Without removing the cartridge from the FEP bag, the FEP bag can be positioned so that the microbe coated side of the carrier is facing the path UVC light. The UVC light can be turned on and the test devices can be exposed to 5 minutes and 10 minutes of UVC light at 5 feet from the light source. Following exposure to UVC light, the carrier can be removed from the FEP bag and the microbes can be recovered by placing the carrier into a container with 5 mL of letheen broth. The container can be vortexed to remove the microbes from the carrier and a sample may be plated onto trypticase soy agar (TSA) and incubated for approximately 24 hours at 35 C.

TABLE 1
Expected Results (Recovered CFU on TSA)

| | Exposure time at distance of 5 feet | |
|---|---|---|
| Replicate | 5 min. | 10 min. |
| 1 | 1.5 × 10\hat{ }3 | 0 |
| 2 | 3.0 × 10\hat{ }3 | 0 |
| 3 | 2.5 × 10\hat{ }3 | 0 |

### Prophetic Example 2—FEP Injection Molded Device

A device can be injection molded out of fluorinated ethylene propylene (FEP). This device can have dimensions of approximately 1″×1″×2″ (L×W×H) and a polypropylene cap, with a small vent (5 mm) and a fabric that covers the vent hole, that fits on one end of the formed part.

Staphylococcus aureus (ATCC 6538) can be grown in synthetic broth for approximately 24 hours at 35° C. S. aureus cells can be diluted into a sterile coating solution containing sucrose (0.4% w/v), ascorbic acid (0.5%), casamino acids (0.6%), and triton X100 (0.04%) to yield approximately 3.0×10\hat{ }6 CFU/carrier. The coating solution can then be spotted into the base of the injection molded part by spotting 10, 1 μL droplets in an array pattern over the surface of the carrier. The coating can then be dried for 10 minutes at atmospheric pressure and approximately 20% relative humidity.

The base of the device can then be capped and placed in a mylar foil pouch with a desiccant and oxygen scavenger and stored at 2-8 C.

At the point of UV testing, the mylar pouch may be removed from 2-8° C. storage. The mylar foil pouch can be opened and the device can be removed from the mylar foil pouch. The device can be positioned so that the microbe coated side is facing the path UVC light. The UVC light can be turned on and the test devices can be exposed to 5 minutes and 10 minutes of UVC light at 5 feet from the light source. Following exposure to UVC light, 2 mL of growth media (TSB) can be aseptically added to the base of the device and incubated for approximately 24 hours at 35 C.

TABLE 1
Expected Results (Positive or negative growth)

| | Exposure time at distance of 5 feet | |
|---|---|---|
| Replicate | 5 min. | 10 min. |
| 1 | Positive | Negative |
| 2 | Positive | Negative |
| 3 | Positive | Negative |

### Example 3—Comparison of FEP, ETFE, and Polystyrene as UV Transmissible Film Using Preserved Staphylococcus aureus (ATCC 6538) Test Carriers as the Microbial Challenge

Staphylococcus aureus (ATCC 6538) was grown in synthetic broth for approximately 24 hours at 35° C. S. aureus cells were then resuspended in sterile coating solution containing sucrose (0.5% w/v), phosphate buffered saline, and 5% v/v fetal bovine serum albumin (FBS) to yield approximately 3.0×10\hat{ }6 CFU/carrier. The coating solution was then spotted onto a sterile glass carrier (1″×1″) by spotting one 10 μL droplet onto the surface of the carrier. The droplet was then spread over the surface of the carrier to within approximately 1 mm of the carrier edges. The carriers were then dried for 35 minutes in a 35° C. incubator. The dried carriers were then placed into a polypropylene slide mailer to hold the slides in place. The slide mailer was then placed inside of a mylar foil pouch containing 10 g silica desiccant and 500 cc oxygen scavenger. The packaged carriers were then stored at 2-8° C. until use.

On the test day, the preserved S. aureus test carriers were removed from the packaging and the mailer and placed in duplicate into the lid of sterile contact plate petri dishes. Four different configurations were prepared with the following materials.
1. No cover control
2. Polystyrene cover—used base of contact plate to cover the test carriers
3. FEP (Fluorinated ethylene propylene (0.001″ thick))—cut piece of film to cover the test carriers
4. ETFE Film (Ethylene tetrafluoroethylene (0.001″ thick)—cut piece of film to cover the test carriers

The prepared test articles were then placed inside of a Nuair Biosafety cabinet and oriented approximately 15 inches from the UV lamp source with the coated microbes facing the lamp source and the test films in-between the UV light and the coated microbes. The samples were exposed for approximately 20 minutes. After exposure, the carriers were aseptically placed into 20 mL of sterile phosphate buffered saline (PBS) and vortexed for 30 seconds each to recover the microbes from the surface of the carriers. The samples were then plated onto trypticase soy agar (TSA) and incubated to determine the colony forming units (CFUs) (Table 3). In addition, an aliquot (200 μL) of each sample was placed into 800 μL of TSB and incubated for approximately 24 hours at 35° C. for a turbidity determination (Table 4).

TABLE 3
Result for CFUs recovered for each sample.

| | | Sample Dilution and volume plated on TSA | | | |
|---|---|---|---|---|---|
| Sample | Barrier | No dilution | 10\hat{ }−1 | 10\hat{ }−1 | 10\hat{ }−2 |
| # | Material | (10 μL) | (50 μL) | (10 μL) | (10 μL) |
| 1 | No cover | 0 | 0 | 0 | 0 |
| 1 | No cover | 0 | 0 | 0 | 0 |
| 2 | Polystyrene | TNTC* | 119 | 22 | 2 |
| 2 | Polystyrene | TNTC* | 111 | 15 | 1 |
| 3 | FEP | 0 | 0 | 0 | 0 |
| 3 | FEP | 0 | 0 | 0 | 0 |
| 4 | ETFE | 0 | 0 | 0 | 0 |
| 4 | ETFE | 0 | 0 | 0 | 0 |

*TNTC = Too numerous to count.

TABLE 4
Result for turbidity or growth for each sample.

| Sample | | | |
|---|---|---|---|
| # | Barrier Material | Growth (+/−) | OD600 |
| 1 | No cover | − | 0 |
| 1 | No cover | − | NA |
| 2 | Polystyrene | + | 0.274 |
| 2 | Polystyrene | + | NA |
| 3 | FEP | − | 0 |
| 3 | FEP | − | NA |
| 4 | ETFE | − | 0 |
| 4 | ETFE | − | NA |

### Example 4

A self-contained biological UV test device was prepared in the following way. Flat polycarbonate carries (8 mm×16 mm×1.5 mm) were prepared. FEP tubing (9 mm O.D. and 8 mm I.D.) was cut into 2.75″ sections. A vinyl cap was placed on one end of the cut tubing and a sterile glass ampoule containing TSB, sucrose, and bromocresol purple was placed into the cut tubing with the cap on one end. PTFE rod was cut into 0.5″ and placed inside of another vinyl cap. These components and assemblies were then steam sterilized for 30 min. at 121° C. in a gravity cycle.

The sterile polycarbonate carriers were then coated with the microbial challenge suspension. The challenge suspension included sucrose (1%), Fetal bovine serum (0.5%), and 0.04% hydrogenated triton X-100 and Bacillus subtilis spores at a concentration to yield>1000 CFU/carrier. Three discrete 2.5 μL spots of the microbial challenge suspension were then placed on each carrier. The carriers were then placed in a vacuum drier for 20 minutes.

The microbial coated carriers were then aseptically placed into the cut FEP tubing assembly with the glass ampoule. The PTFE rod and vinyl cap assembly was then aseptically placed on the open end of the FEP tubing.

A plastic clip, which included one side that partially wraps around the FEP tube, and another side that is flat with an adhesive backing, was then adhered to the FEP tubing using a plastic bonding material. The clip was oriented to enable the fully assembled device to be adhered to a surface using the flat adhesive backing and to allow the microbial coated carrier to face away from the surface and towards the UV light source.

The fully assembled device was then placed inside of a mylar foil pouch containing 1 g silica desiccant and 50 cc oxygen scavenger. The pouch was then stored at room temperature until the test date.

Following storage, the assembled UV test device was removed from the mylar foil pouch. The adhesive backing was removed from the flat portion of the clip, the device was then adhered to the test surface X feet away from the UV light source. Multiple sets of UV test devices were then exposed to different UV doses by varying time of exposure. Following exposure, the UV test devices were activated by breaking the glass ampoule and inverting the device so that the carrier side of the device was facing down and the ampoule media made contact with the microbial coated carrier. The device was then incubated at 35° C. for 18-24 hours. The growth results were then recorded based on the color of the ampoule media (Yellow=growth+, purple=growth−), see Table 5.

TABLE 5
Growth results following exposure.

| Exposure Time | UV Dose | Growth |
|---|---|---|
| (Min.) | (mj/cm2) | Result |
| 2 | 22.44 | + |
| 2 | 22.44 | + |
| 4 | 44.88 | + |
| 4 | 44.88 | − |
| 6 | 67.32 | − |
| 6 | 67.32 | − |

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An ultraviolet (UV) light test device, comprising:
a population of viable microbial material having a residual water content of less than five percent; and
a container encapsulating the population of viable microbial material, the container formed of a material that is microbe impermeable, at least a portion of the container formed of a UV light transmissible material.

2. The UV light test device of claim 1, further comprising an outer package encompassing the container, the outer package formed of an oxygen impermeable material.

3. The UV light test device of claim 1, wherein the population of viable microbial material comprises between 3 to 9 logs of viable microbial material.

4. The UV light test device of claim 1, wherein the population of viable microbial material has a loss of viability of less than 0.5 logs over at least 30 days when stored at a temperature of 4° C.

5. The UV light test device of claim 1, wherein the population of viable microbial material includes bacterial cells or fungal cells.

6. The UV light test device of claim 1, further comprising a substrate bearing the population of viable microbial material disposed on the substrate.

7. The UV light test device of claim 6, wherein the substrate comprises a flat surface with a surface area between 10 mm² and 10,000 mm².

8. The UV light test device of claim 1, wherein the population of viable microbial material comprises a stabilizing material.

9. The UV light test device of claim 8, wherein the stabilizing material comprises a sugar.

10. The UV light test device of claim 8, wherein the stabilizing material comprises an antioxidant.

11. The UV light test device of claim 8, wherein the stabilizing material comprises a growth nutrient.

12. An ultraviolet (UV) light test device, comprising:
a first compartment including a population of viable microbial material disposed on a substrate secured within the first compartment, the population of viable microbial material having a residual water content of less than five percent;
a UV light transmissible cover extending over the first compartment such that a UV light from a UV light source is able to contact the population of viable microbial material disposed on the substrate;
a second compartment sealed from the first compartment, the second compartment including a liquid growth media;
a barrier separating the first compartment and the second compartment, the barrier adapted to be broken after the population of viable microbial material has been exposed to the UV light, the liquid growth media stored within the second compartment able to reach the population of population of viable microbial material once the barrier is broken.

13. The UV light test device of claim 12, wherein the barrier comprises a frangible member.

14. The UV light test device of claim 12, further comprising an indicator that provides an indication of whether there is residual viable microbial material after the population of viable microbial material has been exposed to the UV light and contacted with the liquid growth media.

15. The UV light test device of claim 12, wherein the population of viable microbial material comprises between 3 to 9 logs of viable microbial material.

16. The UV light test device of claim 12, wherein the population of viable microbial material has a loss of viability of less than 0.5 logs over at least 30 days when stored at a temperature of 4° C.

17. The UV light test device of claim 12, wherein the population of viable microbial material includes bacterial cells or fungal cells.

18. The UV light test device of claim 12, wherein the substrate comprises a flat surface with a surface area between 10 mm² and 10,000 mm².

19. A method of evaluating the efficacy of a germicidal light, comprising:
removing a germicidal light test device from a sealed container,
securing the germicidal light test device in a fixed orientation
exposing the germicidal light test device to a germicidal light for a duration of time and at a certain distance,
removing the germicidal light test device from the fixed orientation, and
performing an analysis to determine the effectiveness of the germicidal light.

20. The method of claim 19, wherein the biological material is contained within the germicidal light test device and is not exposed to the environment.
